(19) United States
(12) Patent Application Publication
(10) Pub. No.: US 2023/0013889 A1
(43) Pub. Date: Jan. 19, 2023

(54) FILAMENT FOR PRODUCING TWO-WAY LIVE COMMUNICATION USING AUDIO, VIDEO, PICTURE AND TEXT MESSAGES FROM MOBILE DEVICES TO SMART/STANDARD TELEVISIONS

(71) Applicant: Delandia Deverne Dakin, New York, NY (US)

(72) Inventor: Delandia Deverne Dakin, New York, NY (US)

(21) Appl. No.: 17/305,889

(22) Filed: Jul. 16, 2021

(51) Int. Cl. G16H 40/67; H04N 7/15; H04N 7/14; G16H 80/00; G16H 70/20

(52) U.S. Cl. CPC G16H 40/67; H04N 7/152; H04N 7/147; G16H 80/00; G16H 70/20

(57) ABSTRACT

A filament for producing a two-way live communication system using audio, video, picture, and text messages from a patient's television in hospitable environments to a distant love one's mobile device. The present invention includes software that enables patients and remote loved ones and caregivers to remain in consistent contact especially during crisis situations with the downloading of the application on both the patient's television and mobile devices. Upon downloading this software each member is prompted to enter in their required information in order to connect with the individual they seek. The software will also be used by doctors to communicate with a patient's primary care family member, that patient's pending treatments and goals. The software is built to expand communication channel on a tool which over 95% of individuals have access to and watch daily.

### BACKGROUND OF THE INVENTION

The present invention relates to communication systems and software developments, more particularly a software all-in-one development that allows for two-way communication from a mobile device to smart and standard television that will display modules of telecommunication via video, audio, text and picture messages, including frequency links as such, and text messages via standardized communication protocols and/or videotelephony as well as other technology harnessing audio-video signals.

Those in long term care facilities, to take one exemplary instance, do not have the time nor perhaps the faculties to take advantage of current technology to control the multiple communication media that could substantially enhance their lives. According to research those in long term care facilities who do have access to cellular phones, only 20% of these patients were able to properly use such devices, though over 95% of such residents may have access to a smart or standardized television. Currently there are no developments within this field created to be used in this manner for two-way family/friend and professional communication.

The present invention was created to connect to smart and standard televisions in such a way as to enable bidirectional transmissions of live text, call, picture, video messages and other audio-video signals embodied in the current state of the communication arts.

Software developments and healthcare fields go hand in hand with cutting edge technologies. From transformational advances to breakthrough discoveries, software developments in the healthcare systems rely heavily on innovations. Constance Artistry Care LLC's “CAC software”, for instance, is a wireless interactive software program specifically designed for the healthcare sector. CAC software represents an innovative approach to improving the quality of communication amongst patients, the professionals, and the loved ones invested in the patient's care. The present invention creates another way to be in consistent contact on something that every (client) patient has in their rooms—television—over wireless communication, such as the use of WIFI™.

Furthermore, the present invention is a solution to a large problem that many face as has become apparent with the onset of Covid-19. The healthcare system failed to meet the needs of millions due to the lack of innovative technologies. It has been shown that the healthcare system can improve with technological developments, and future technological innovation is going to continue transforming the world. It has also been proven that human interactions improve one's health for those who are in long term care facilities. While life became extremely challenging due to Covid-19 more individuals were seeking technical resources to complete their daily task, improve their time management, and adapt to the never-seen-before changes. The world shut down, but the healthcare field became busier, and most importantly visitation was completely closed off to public visitators. CAC software's resilient healthcare innovation is adapted to help people with technology.

Specifically, the present invention is adapted to bring back familiar interactions between patient and family members, while also improving the overall dynamics that the professionals in the long-term care facilities use to improve their patient's wellbeing. The expectation of CAC software is to execute the aforementioned functionality as well as encourage dialogue via text, video, audio and pictures messages from a mobile device to smart/standardized televisions, thereby facilitating collaboration between all parties involved with the sole purpose of sustaining one's wellbeing. CAC software finds that technology combined with human interaction will have major health benefits. The healthcare software of the present invention is an innovative service in this manner due to combining these components.

### SUMMARY OF THE INVENTION

In one aspect of the present invention provides an improvement to how a television is used and includes the following: a sender/receiver of text, picture, audio and video files to a mobile device with the download of the application and access to wireless communication. The other aspect of the present invention is mobile devices being enabled to send and receive video, picture, audio and text messages to and from a smart/standard television with the download of the CAC software over WIFI or other routes of connections to the Internet. The cellular phone module and television may be configured through a software application to represent one or more audio-visual communications. The improvement to a television, in short, is the installment of a software application onto the TV; and a cellular phone module electrically connected to the software application, wherein the cellular phone module is configured through a software application to represent one or more audio-visual files via the television, wherein the software application represents the one or more audio-visual files on a display portion of a visual display of the television, wherein if needed the software application is downloaded on a remote device so as to configure the remote device such as a comcast or firestick to enable bidirectional communication with the television.

In another aspect of the present invention, this innovation addresses the barriers facing the chronically ill, the lack of consistent communication and time spent with their distant loved ones, and the limited technological resources provided to them. This business innovation also addresses the ways in which facilities can use this software to enhance their time management and protect themselves as well as their patients, while also allowing for them to properly conduct their jobs during any situation but more importantly during a crisis. The method includes electrically connecting the above-mentioned improvement on a mobile device to a television of said resident using the CAC software; registering one or more first parties through the software with a cellular phone module or on the TV; and transmitting, by the one or more first parties, the one or more audio-visual files to the television, wherein the one or more first parties include family members, friends or a medical practitioner, and wherein the one or more audio-visual files includes pictures, videos, text, and audios sent from a distant love one or a medical treatment plan from the medical practitioner.

These and other features, aspects and advantages of the present invention will become better understood with reference to the following drawings, description and claims.

### BRIEF DESCRIPTION OF THE DRAWINGS

FIG. 1 is a schematic view of an exemplary embodiment of the present invention; and

FIG. 2 is a flow chart of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, an embodiment of the present invention provides a communication system incorporating a single software/application development through a mobile device and a smart or standard television with wireless access, both configured to send and receive voice audios, video, text and picture messages.

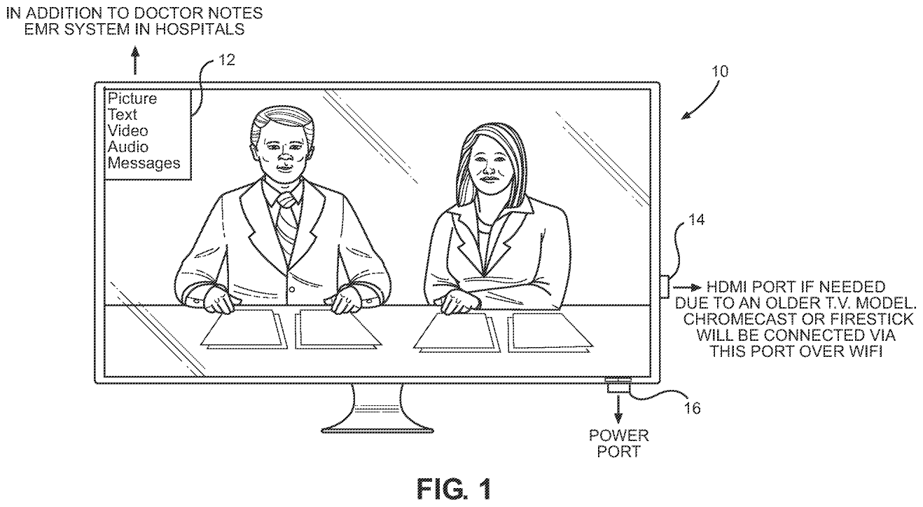

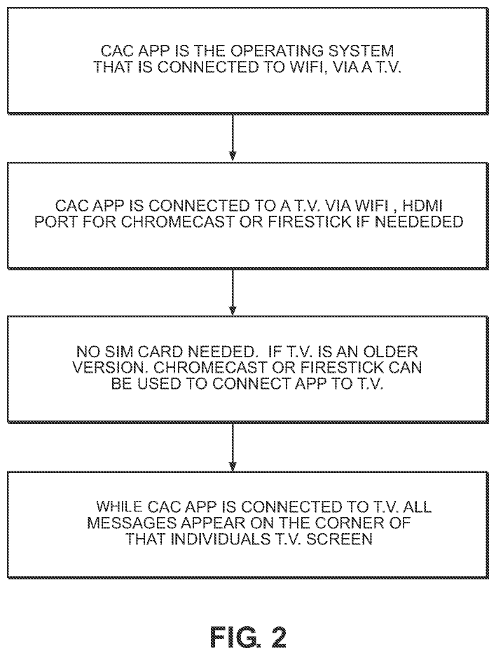

Referring now to FIGS. 1 and 2, the present invention may include a communication system incorporating a television and all-in-one cellular phone module configured through a software application (hereafter, “CAC software”), to add voice audios, text, and video data to a smart or standard television 10. The all-in-one cellular phone module may include, but is not limited to, a particular cellular device or the like. The television and all-in-one cellular phone module may be configured by the CAC software application and an older standardized television model may need access to a firestick or comcast via the HDMI port 14 and wireless communication protocols or direct internet access. The smart or standard television 10 just needs a power source 16 to represent voice, text, video, and picture data on a display portion 12 of the visual display of the television 10. It should be understood that the display portion 12 may be any shape and may be located along any area of the visual display (not just the upper left corner as shown in FIG. 1) such as the upper right corner, along the lower edge, or the like.

A user may download the CAC software and sign up for a membership plan. Following this step, the first party must enter in their personal information along with a second party's (e.g., a patient's) information room number and code linked to the patient's cellular phone module which is electrically connected to the second party's television 10. Once information is entered the first party user may begin to send recorded videos, audios, picture and text messages that are transmitted to the second party television 10—the messages may then pop up directly along a portion of the a display portion of the second party's smart or standard television 10, while they are watching the television 10.

The CAC software is a development that enables two-way communication from a television to mobile device. Once these two devices are connected the first party and the second party may be able to electronically communicate via the visual display of the television 10 of the second party, enabling the first party to perform the functions of recording live video and audio messages, sending picture and written text messages that appear directly on that second party's television 10 display portion.

For older televisions, digital media players and/or wireless hardware may be required. A membership identification module (for instance, “the CAC's SOFTWARE CODE”) will also be required.

The present invention allows for patients to communicate with their distant love ones. Professionals in this field now have a reliable resource of communication during a crisis situation to communicate with the patient's primary caregiver and vise versa for the family member to the professional with the use of sending audios, videos, and other messages to their friends or family members who are sick in long term care facilities. These messages will populate on the second party's patient's TV screen.

The role of the CAC software is to provide an affordable software development to the patients, their family members and the professionals that offers a direct streamline of concise communication. The CAC software is unique and transparent in its route/methodoly used for this two-way communication which derives from the patient's TV to the distant loved one's mobile device. This business model takes into account the health improvements for a patient when being in contact with their distant love ones, greater time management for the family and friends and the innovative resources provided to the professionals to improve on their quality or work.

Additionally, the present invention also creates a way for patients to respond to audios, messages, videos and pictures using their remote control which is connected to their television or other audio-visual communication technology, such as, but not limited to, television, videotelephony, teleconferencing and other systems.

In certain embodiments, the network may refer to any interconnecting system capable of transmitting audio, video, signals, data, messages, or any combination of the preceding. The network may include all or a portion of a public switched telephone network (PSTN), a public or private data network, a local area network (LAN), a metropolitan area network (MAN), a wide area network (WAN), a local, regional, or global communication or computer network such as the Internet, a wireline or wireless network, an enterprise intranet, or any other suitable communication link, including combinations thereof.

The server and the television/computer of the present invention may each include computing systems. This disclosure contemplates any suitable number of computing systems. This disclosure contemplates the computing system taking any suitable physical form. As example and not by way of limitation, the computing system may be a virtual machine (VM), an embedded computing system, a system-on-chip (SOC), a single-board computing system (SBC) (e.g., a computer-on-module (COM) or system-on-module (SOM)), a desktop computing system, a laptop or notebook computing system, a smart phone, an interactive kiosk, a mainframe, a mesh of computing systems, a server, an application server, or a combination of two or more of these. Where appropriate, the computing systems may include one or more computing systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example and not by way of limitation, one or more computing systems may perform in real time or in batch mode one or more steps of one or more methods described or illustrated herein. One or more computing systems may perform at different times or at different locations one or more steps of one or more methods described or illustrated herein, where appropriate.

In some embodiments, the computing systems may execute any suitable operating system such as IBM's zSeries/Operating System (z/OS), MS-DOS, PC-DOS, MAC-OS, WINDOWS, UNIX, OpenVMS, an operating system based on LINUX, or any other appropriate operating system, including future operating systems. In some embodiments, the computing systems may be a web server running web server applications such as Apache, Microsoft's Internet Information Server™, and the like.

In particular embodiments, the computing systems includes a processor, a memory, a user interface and a communication interface. In particular embodiments, the processor includes hardware for executing instructions, such as those making up a computer program. The memory includes main memory for storing instructions such as computer program(s) for the processor to execute, or data for processor to operate on. The memory may include mass storage for data and instructions such as the computer program. As an example, and not by way of limitation, the memory may include an HDD, a floppy disk drive, flash memory, an optical disc, a magneto-optical disc, magnetic tape, a Universal Serial Bus (USB) drive, a solid-state drive (SSD), or a combination of two or more of these. The memory may include removable or non-removable (or fixed) media, where appropriate. The memory may be internal or external to computing system, where appropriate. In particular embodiments, the memory is non-volatile, solid-state memory.

The user interface includes hardware, software, or both providing one or more interfaces for communication between a person and the computer systems. As an example, and not by way of limitation, an user interface device may include a keyboard, keypad, microphone, monitor, mouse, printer, scanner, speaker, still camera, stylus, tablet, touchscreen, trackball, video camera, another suitable user interface or a combination of two or more of these. A user interface may include one or more sensors. This disclosure contemplates any suitable user interface and any suitable user interfaces for them.

The communication interface includes hardware, software, or both providing one or more interfaces for communication (e.g., packet-based communication) between the computing systems over the network. As an example, and not by way of limitation, the communication interface may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface. As an example and not by way of limitation, the computing systems may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the computing systems may communicate with a wireless PAN (WPAN) (e.g., a BLUETOOTH WPAN), a WI-FI network, a WI-MAX network, a cellular telephone network (e.g., a Global System for Mobile Communications (GSM) network), or other suitable wireless network or a combination of two or more of these. The computing systems may include any suitable communication interface for any of these networks, where appropriate.

The present invention may include a communication system or filament for incorporating a single-board computer and all-in-one cellular phone module configured through the CAC software application to add voice, video, audio and text, and data to a standard television 10. The all-in-one cellular phone module may include, but is not limited to, smart phones or the like. The single-board computer and all-in-one cellular phone module may be configured by the CAC software to use a HDMI port 14 and/or wireless communication protocols or direct internet access to enable the disclosure herein.

A user may download the CAC software and register or enter first party information along with second party's (e.g., a patient's) information and number linked to the single-board computer and all-in-one cellular phone module which are electrically connected to the second party's television 10. Once information is entered the first party user may begin to record a video, send texts, and picture messages, record an audio and/or video messages that are transmitted to the second party television 10—the messages may then pop up directly in along a portion of the second party's standard television 10, while they are watching the smart or standard television 10.

The single-board computer connects directly to a standard television, providing a mother board which can operate as a mini-computer, cell phone or alarm clock. Of course, it is understood, the single-board computer could be a multi-board computing device in some embodiments. The all-in-one cellular phone module may be the other component that is connected to the single-board computer which operates as a cell phone. Once these two devices are connected the first party and the second party may be able to electronically communicate via the visual display of the television 10 of the second party, enabling the first party to perform the functions of making phone calls, recording live video and audio messages, sending picture and written text messages that appear directly on a portion of that second party's television 10 visual display.

It should be understood, of course, that the foregoing relates to exemplary embodiments of the invention and that modifications may be made without departing from the spirit and scope of the invention as set forth in the following claims.

What is claimed is:

1. An improvement to a television, comprising:
a software development; and
a cellular phone module electrically connected to the software development, wherein the cellular phone module is configured through a software application to represent one or more audio-visual files via the television.

2. The improvement to a television of claim 1, further comprising wherein the software application represents the one or more audio-visual files on a display portion of a visual display of the television.

3. The improvement to a television of claim 2, wherein the software application is downloaded on a remote device so as to configure the remote device to enable bidirectional communication with the television.

4. A method of enhancing a quality of communication of a resident in long-term care facilities, the method comprising:
electrically connecting the improvement for a television of claim 3 to a television of said resident;
registering one or more first parties with said cellular phone module; and
transmitting, by the one or more first parties, the one or more audio-visual files to the television.

5. The method of claim 4, wherein the one or more first parties include a medical practitioner, and wherein the one or more audio-visual files includes medical treatment plan from the medical practitioner.
